(19) United States
(12) Patent Application Publication
(10) Pub. No.: US 2021/0369753 A1
(43) Pub. Date: Dec. 2, 2021

(54) NUTRIENT COMPOSITION ABLE TO INHIBIT QUORUM SENSING

(71) Applicant: Brilliant Lab LLC, American Fork, UT (US)

(72) Inventors: Daniel A. Gubler, Orem, UT (US); Michael Saunders, American Fork, UT (US)

(21) Appl. No.: 17/240,834

(22) Filed: Apr. 26, 2021

Related U.S. Application Data
(60) Provisional application No. 63/030,766, filed on May 27, 2020.

(51) Int. Cl. A61K 31/7048; A61K 36/61; A61K 31/26; A61K 36/185; A61K 31/216; A61K 31/353; A61K 35/644; A61K 36/23; A61K 36/9064; A61K 36/258; A61K 31/11; A61K 31/235; A61K 35/20; A61K 31/715; A61K 38/40; A61K 33/30; A61K 9/48; A61P 37/04; A61P 31/04

(52) U.S. Cl. CPC A61K 31/7048; A61P 31/04; A61K 31/26; A61K 36/185; A61K 31/216; A61K 31/353; A61K 35/644; A61K 36/23; A61K 36/9064; A61K 36/258; A61K 31/11; A61K 31/235; A61K 35/20; A61K 31/715; A61K 38/40; A61K 33/30; A61K 9/4825; A61P 37/04; A61K 36/61

(57) ABSTRACT

Described, among other things, are dosage forms for administration to a subject, the dosage forms having at least six ingredients are selected are from the group consisting of clove powder, rutin, hesperidin, baicalin, sulforaphane, moringa leaf extract, rosmarinic acid, guava leaf extract, Panax notoginseng, EGCG, propolis, coriander extract, cardamom extract, cinnamaldehyde, chlorogenic acid, caper fruit, vescalagin, nutmeg extract, and naringenin. The dosage forms can serve as inhibitors of quorum sensing by infectious agents. The described combinations of selected natural products have the ability to inhibit quorum sensing. Quorum sensing is a means by which bacteria and viruses communicate with each other and induce virulence in the body. This combination of natural products is preferably included in nutraceutical formulations in order to promote human immune health.

### CROSS-REFERENCE TO RELATED APPLICATION

This application claims the benefit under 35 U.S.C. § 119(e) of U.S. Provisional Patent Application Ser. No. 63/030,766, filed May 27, 2020, the disclosure of which is hereby incorporated herein in its entirety by this reference.

### TECHNICAL FIELD

The application relates generally to nutritional supplements and associated methods of making and using them. More particularly, the applications relates to supplements containing multiple, select nutrients, which do not co-exist in any naturally occurring composition, useful for, among other things, inhibiting quorum sensing in bacteria and viruses.

### BACKGROUND

Bacterial and viral infections are some of the most common maladies of the human condition. These illnesses are so widespread, a large portion of bacterial infections are simply referred to as the “common cold.” These illnesses can range from merely annoying to life threatening.

The body's immune system is the primary system responsible for, first, detecting bacterial and viral invaders and, second, recruiting antibodies and macrophages to destroy these unwelcome guests. Described herein is a unique mode of action able to be targeted to support the health of the immune system and potentially increase its effectiveness to fight off bacteria, fungi, and viruses.

The word “quorum” is used in business, religious, and other organizations to denote a certain concentration of individuals needed to conduct business, make binding decisions, and get work done. “Quorum Sensing” refers to a form of chemical communication whereby microbes (e.g., bacterial cells of the same and/or of different species) can communicate with each other to increase their ability to survive.

Bacterial activities involving quorum sensing include biofilm formation, virulence, motility, bioluminescence, sporulation, and nitrogen fixation. It was observed that bacteria living together in high concentrations had a molecular switch flipped on that activated certain genes—allowing the bacteria to work together to have increased functionality than when they were isolated. Quorum sensing was first discovered in the Hawaiian bobtail squid where it was observed that Vibrio fischeri bacteria, if present in a high enough concentration, could cause bioluminescence in the squid in a symbiotic relationship. Verma et al., “Quorum sensing in the squid-Vibrio symbiosis,” Int. J. Mol. Sci. 14(8):16386-401 (2013); doi: 10.3390/ijms140816386, the contents of which are incorporated herein by this reference. Quorum sensing is a big problem in the medical implant market where biofilm formation on implants and prostheses can cause systemic inflammation that necessitate implant replacement.

Rigorous scientific research found that bacteria emit quorum sensing molecules—either small organic molecules or peptides—that elicit gene activation upon reaching a threshold concentration. It was also discovered that Gram-positive bacteria secrete a small peptide called auto-inducing peptide (“ATP”), while Gram-negative bacteria exude small organic molecules call auto-inducers (“AI”).

### BRIEF SUMMARY

Supporting human immune health via an everyday preventative well-care approach is a core tenant of overall health. Described herein is a unique composition of, e.g., ingredients with the ability to inhibit the quorum sensing process, the process by which, e.g., bacteria communicate with each other (i.e., quorum sensing), become virulent, and compromise health of the immune system. This composition may be taken separately or incorporated into an immune health supplement and promotes health of the immune system.

Also described is a polypharmacological formulation that promotes health of the human immune system by inhibiting quorum sensing of various bacteria and viruses that can compromise health.

Included is a dosage form for administration to a subject, the dosage form comprising at least six ingredients selected from the group consisting of clove powder, rutin, hesperidin, baicalin, sulforaphane, moringa leaf extract, rosmarinic acid, guava leaf extract, Panax notoginseng, EGCG, propolis, coriander extract, cardamom extract, cinnamaldehyde, chlorogenic acid, caper fruit, vescalagin, nutmeg extract, and naringenin. This dosage form will typically, but not necessarily contains amounts of the selected at least six ingredients to inhibit quorum sensing by bacteria or viruses. In certain embodiments, the amounts can be divided between more than one dosage form, and the subject (e.g., a mammal, such as a human) administered the desired amounts in, e.g., two capsules, or, e.g., two or three times a day.

Typically, the dosage contain the selected at least six ingredients, when present, are present in the following amounts: from about 25 to about 200 milligrams of clove powder; from about 30 to about 100 milligrams of rutin; from about 40 to about 80 milligrams of hesperidin; from about 60 to about 100 milligrams of baicalin; from about 0.08 to about 2 milligrams of sulforaphane; from about 50 to about 120 milligrams of moringa leaf extract; from about 4 to about 8 milligrams of rosmarinic acid; from about 10 to about 30 milligrams of guava leaf extract; from about 75 to about 200 milligrams of Panax notoginseng; from about 75 to about 160 milligrams of EGCG; from about 20 to about 40 milligrams propolis; from about 50 to about 90 milligrams of coriander extract; from about 30 to about 60 milligrams of cardamom extract; from about 2 to about 5 milligrams of cinnamaldehyde; from about 50 to about 90 milligrams of chlorogenic acid; from about 20 to about 40 milligrams of caper fruit; from about 3 to about 8 milligrams of vescalagin; from about 10 to about 150 milligrams of nutmeg extract; and from about 10 to about 30 milligrams of naringenin.

These dosage forms can further contain other nutraceutical raw ingredients thought to support health (such as colostrum, arabinogalactan, lactoferrin, zinc, and/or elderberry.)

Disclosed herein is a method of inhibiting quorum sensing by an infectious agent or infectious agents in a subject, the method including administering the described dosage forms to the subject, for example on a once, twice, three, or four time daily basis. The described dosage forms and methods can be used for treatment and/or prophylaxis of infection.

In certain embodiments, the method of inhibiting quorum sensing by an infectious agent in a subject comprises: administering a combination of at least six selected ingredients to the subject, wherein the at least six ingredients are selected are from the group consisting of clove powder, rutin, hesperidin, baicalin, sulforaphane, moringa leaf extract, rosmarinic acid, guava leaf extract, Panax notoginseng, EGCG, propolis, coriander extract, cardamom extract, cinnamaldehyde, chlorogenic acid, caper fruit, vescalagin, nutmeg extract, and naringenin.

Typically, the dosages of the selected ingredient in the dosage form will be sufficient to inhibit quorum sensing by the infectious agent. For example, in such a method, the subject ingests the selected at least six ingredients, when selected, in the following amounts on a daily basis: from about 25 to about 200 milligrams of clove powder, from about 30 to about 100 milligrams of rutin, from about 40 to about 80 milligrams of hesperidin, from about 60 to about 100 milligrams of baicalin, from about 0.08 to about 2 milligrams of sulforaphane, from about 50 to about 120 milligrams of moringa leaf extract, from about 4 to about 8 milligrams of rosmarinic acid, from about 10 to about 30 milligrams of guava leaf extract, from about 75 to about 200 milligrams of Panax notoginseng, from about 75 to about 160 milligrams of EGCG, from about 20 to about 40 milligrams propolis, from about 50 to about 90 milligrams of coriander extract, from about 30 to about 60 milligrams of cardamom extract, from about 2 to about 5 milligrams of cinnamaldehyde, from about 50 to about 90 milligrams of chlorogenic acid, from about 20 to about 40 milligrams of caper fruit, from about 3 to about 8 milligrams of vescalagin, from about 10 to about 150 milligrams of nutmeg extract and from about 10 to about 30 milligrams of naringenin.

Such methods may be used to inhibit quorum sensing by an infectious agent selected from the group consisting of bacteria, viruses, fungi, protozoa, and helminths. A typical infectious agent is a bacterium selected from the group consisting of Pseudomonas aeruginosa, Escherichia coli, Chromobacterium violaceum, Pseudomonas fluorescens, Staphylococcus aureus, Helicobacter pylori, Candida albicans, Streptococcus mutans, Bacillus cereus, Proteus mirabilis, Serratia marcescens, Acinetobacter, Burkholderia, and any combination thereof. Preferably, the composition of ingredients including plant secondary metabolites will have the ability to inhibit quorum sensing in both bacteria and viruses responsible for causing disease in humans.

While not intending to be bound by theory, the following may help to explain the invention. Quorum sensing is intimately involved in, for example, the process of bacteria becoming virulent—or capable of both infecting and damaging the host. Disease-causing bacteria can and do flow harmlessly in and out of the body without becoming virulent. Virulence occurs when the AHL auto-inducer concentration increases to the point that genes in bacteria (e.g., S. mutans) are turned on that increase their capacity to infect and damage the host organism. This effect provides an avenue for supporting immune health. In short, if one were to keep auto-inducer concentrations low, one would be able to stop quorum sensing from occurring regardless of bacterial cell density. Additionally, viruses use the same principle of quorum sensing (consider, e.g., arbitrium in bacteriophage) in order to communicate and coordinate virulence viruses. See, e.g., Dolgin, Elie “The secret social lives of viruses,” Nature 2019 570 (7761): 290-292. doi:10.1038/d41586-019-01880-6, the contents of which are incorporated herein by this reference.

These inhibitors work by different mechanisms to inhibit the message produced by natural auto-inducers, prevent the infectious agents from communicating with one another, and keep various quorum sensing related genes turned off. Thus, using combinations of the six different inhibitors of quorum sensing each inhibitor having a different mechanism and antimicrobial spectrum of activity (as described herein) acts to inhibit quorum sensing by infectious agents and/or avoid resistance emergence in a manner greater than the individual components.

### DETAILED DESCRIPTION

The described dosage form comprises at least six ingredients selected from the group consisting of clove powder, rutin, hesperidin, baicalin, sulforaphane, moringa leaf extract, rosmarinic acid, guava leaf extract, Panax notoginseng, EGCG, propolis, coriander extract, cardamom extract, cinnamaldehyde, chlorogenic acid, caper fruit, vescalagin, nutmeg extract, and naringenin.

Clove powder is readily commercially available and inhibits quorum sensing in P. aeruginosa PAO1, E. coli [pSB401] and [pSB1075]. It is typically utilized in in a dosage form hereof at a dosage of about (plus or minus 5%) 25 to about 200 mg.

Rutin are found in the rind of green citrus fruits and in rose hips and black currants. Rutin is readily commercially available and inhibits quorum sensing in E. coli. Rutin is typically utilized in in a dosage form hereof at a dosage of about (plus or minus 5%) 30 to about 100 mg.

Hesperidin is a flavanone glycoside found in citrus fruits. Hesperidin is readily commercially available and inhibits quorum sensing in C. violaceum and P. fluorescens. It is typically utilized in in a dosage form hereof at a dosage of about (plus or minus 5%) 40 to about 80 mg.

Baicalin is a flavone glycoside. Baicalin is readily commercially available and inhibits quorum sensing in C. violaceum CV026 and S. aureus. It is typically utilized in in a dosage form hereof at a dosage of about (plus or minus 5%) 60 to about 100 mg.

Sulforaphane is a compound within the isothiocyanate group of organosulfur compounds. It is obtained from cruciferous vegetables such as broccoli, Brussels sprouts, and cabbage. Sulforaphane is readily commercially available and inhibits quorum sensing in P. aeruginosa. It is typically utilized in in a dosage form hereof at a dosage of about (plus or minus 5%) 0.08 to about 2 mg.

Moringa leaf extract comes from Moringa oleifera, a fairly large tree (drumstick tree, horseradish tree or ben oil tree) native to India. Moringa leaf extract is readily commercially available and inhibits quorum sensing in C. violaceum (ATCC 12472). It is typically utilized in in a dosage form hereof at a dosage of about (plus or minus 5%) 50 to about 120 mg.

Rosmarinic Acid is a polyphenol similar to caffeic acid (found in coffee) and is in high levels in perilla oil and rosemary. Rosmarinic acid is readily commercially available and inhibits quorum sensing in C. violaceum CV026, C. violaceum 31532, P. aeruginosa PAO1, and E. coli O157:H7. It is typically utilized in in a dosage form hereof at a dosage of about (plus or minus 5%) 4 to about 8 mg.

Guava leaf extract is readily commercially available and inhibits quorum sensing in C. violaceum. It is typically utilized in in a dosage form hereof at a dosage of about (plus or minus 5%) 10 to about 30 mg.

Panax notoginseng (“Chinese ginseng”) is readily commercially available and inhibits quorum sensing in P. aeruginosa, C. violaceum, H. pylori, S. aureus, E. coli, and C. albicans. It is typically utilized in in a dosage form hereof at a dosage of about (plus or minus 5%) 75 to about 200 mg.

EGCG is the most abundant catechin in tea, and is a polyphenol. EGCG is readily commercially available and inhibits quorum sensing in P. aeruginosa. It is typically utilized in in a dosage form hereof at a dosage of about (plus or minus 5%) 75 to about 160 mg.

Propolis (“bee glue”) is a resinous mixture that honey bees produce by mixing saliva and beeswax with exudate gathered from tree buds, sap flows, or other botanical sources. Propolis is readily commercially available and inhibits quorum sensing in C. violaceum. It is typically utilized in in a dosage form hereof at a dosage of about (plus or minus 5%) 20 to about 40 mg.

Coriander extract is readily commercially available and inhibits quorum sensing in E. coli, S. enterica, P. aeruginosa, and S. aureus. It is typically utilized in in a dosage form hereof at a dosage of about (plus or minus 5%) 50 to about 90 mg.

Cardamom extract is readily commercially available and inhibits quorum sensing in C. albicans, S. mutans, S. aureus, and B. cereus. It is typically utilized in in a dosage form hereof at a dosage of about (plus or minus 5%) 30-60 mg.

Cinnamaldehyde is the aldehyde that gives cinnamon its flavor and odor. Cinnamaldehyde occurs naturally in the bark of cinnamon trees and other species of the genus Cinnamomum, such as camphor and cassia. Cinnamaldehyde is readily commercially available and inhibits quorum sensing in P. aeruginosa PAO1, and C. violaceum CV026. It is typically utilized in in a dosage form hereof at a dosage of about (plus or minus 5%) 2 to about 5 mg.

Chlorogenic acid is a compound found in a wide variety of foods and beverages, including fruits, vegetables, olive oil, spices, wine, and coffee. Chlorogenic acid is readily commercially available and inhibits quorum sensing in P. aeruginosa. It is typically utilized in in a dosage form hereof at a dosage of about (plus or minus 5%) 10 to about 20 mg.

Caper fruit (or caper flowers) is readily commercially available and inhibits quorum sensing in C. violaceum CV026, P. aeruginosa, E. coli, P. mirabilis, and S. marcescens. It is typically utilized in in a dosage form hereof at a dosage of about (plus or minus 5%) 20 to about 40 mg.

Vescalagin is readily commercially available and inhibits quorum sensing in P. aeruginosa, Acinetobacter, and Burkholderia. It is typically utilized in in a dosage form hereof at a dosage of about (plus or minus 5%) 3 to about 8 mg.

Nutmeg extract is readily commercially available and inhibits quorum sensing in P. aeruginosa PAO1, and C. violaceum CV026. It is typically utilized in in a dosage form hereof at a dosage of about (plus or minus 5%) 100 to about 150 mg.

Naringenin is a flavorless, colorless flavanone, a type of flavonoid. It is the predominant flavanone in grapefruit, and is found in a variety of fruits and herbs. Naringenin is readily commercially available and inhibits quorum sensing in P. aeruginosa PAO1. It is typically utilized in in a dosage form hereof at a dosage of about (plus or minus 5%) 10 to about 30 mg.

In certain embodiments, the at least six ingredients are selected from the group consisting of rutin, hesperidin, baicalin, sulforaphane, rosmarinic acid, cinnamaldehyde, chlorogenic acid, and naringenin. In the case of treatment or prevention of infection by fungal compositions, the dosage form will typically include both Panax notoginseng and cardamom extract in the amounts described herein. In the case of treatment or prevention of infection by P. aeruginosa, the dosage form can contain, e.g., Panax notoginseng, EGCG, coriander extract, cinnamaldehyde, chlorogenic acid, caper fruit, vescalagin, nutmeg extract, and/or naringenin in the amounts described herein. In the case of treatment or prevention of infection by E. coli, the dosage form can contain, e.g., clove powder, rutin, sulforaphane, rosmarinic acid, P. notoginseng, coriander extract, and/or caper fruit in the amounts described herein. In the case of treatment or prevention of infection by S. aureus, the dosage form can contain, e.g., baicalin, P. notoginseng, coriander extract, and/or cardamom extract in the amounts described herein. In the case of treatment or prevention of infection by viruses, the dosage form can contain, e.g., hesperidin, rutin, and/or sulforaphane in the amounts described herein.

The described dosage forms and methods can be used, for example, in addition to standard antibiotic therapy.

Once being apprised of the instant disclosure, a person of ordinary skill in the art will be readily able to make or prepare the dosage forms using Galenical techniques. Preferably, a finished product delivery forms is selected from the group consisting of softgel, capsule, tablet, gel, powder, gummy, liquid, effervescent, bar, topical patch, serum, stick pack, ready to drink composition, lotion, and cream.

The invention is further described with the aid of the following illustrative Examples.

### EXAMPLES

### Example I

The following ingredients are thoroughly admixed as close to uniform consistency as possible:

| | | |
|---|---|---|
| Panax notoginseng | 150 | grams |
| EGCG | 120 | grams |
| Propolis | 30 | grams |
| Coriander extract | 70 | grams |
| Cardamom extract | 45 | grams |
| Cinnamaldehyde | 3 | grams |
| Chlorogenic acid | 70 | grams |
| Caper fruit | 30 | grams |
| Vescalagin | 5 | grams |
| Nutmeg extract | 80 | grams |
| Naringenin | 20 | grams |

The resulting admixture is divided into 1,000 equal portions each placed into one of 1,000 appropriately-sized hard (or vegan) gelatin capsules.

### Example II

Capsules of EXAMPLE I are administered in a dosing regimen of one capsule daily (e.g., with breakfast and dinner) to a subject at risk having recurrent fungal infections. The treatment regimen is continued for 13 weeks. The described supplement supports the subject's health via, for example, a preventative well-care approach, and the candida infection is controlled in the subject.

### Example III

The following ingredients are thoroughly admixed:

| | | |
|---|---|---|
| Panax notoginseng | 100 | grams |
| EGCG | 100 | grams |
| Coriander extract | 75 | grams |
| Cinnamaldehyde | 4 | grams |
| Chlorogenic acid | 80 | grams |
| Caper flower | 30 | grams |
| Vescalagin | 6 | grams |
| Nutmeg extract | 100 | grams |
| Naringenin | 20 | grams |

The resulting admixture is divided into 1,000 equal portions and each portion is placed into one of 1,000 appropriately-sized hard gelatine (or vegan) capsules.

### Example IV

Capsules of EXAMPLE III are administered in a dosing regimen of one capsule daily (e.g., with breakfast and dinner) to a subject at risk for infection by P. aeruginosa. The treatment regimen is continued for 13 weeks.

### Example V

The following ingredients are thoroughly admixed as close to uniform consistency as possible:

| | | |
|---|---|---|
| Clove powder | 100 | grams |
| Rutin | 75 | gram |
| Sulforaphane | 1 | grams |
| Rosmarinic acid | 6 | grams |
| Panax notoginseng | 125 | grams |
| Coriander extract | 70 | grams |
| Caper fruit | 30 | grams |
| Vescalagin | 5 | grams |
| Naringenin | 20 | grams |

The resulting admixture is divided into 1,000 equal portions each placed into one of 1,000 appropriately-sized hard (or vegan) gelatin capsules.

### Example VI

Capsules of EXAMPLE V are administered in a dosing regimen of one capsule daily (e.g., with breakfast and dinner) to a subject at risk for infection by E. coli. The treatment regimen is continued for 26 weeks.

### Example VII

The following ingredients are thoroughly admixed:

| | | |
|---|---|---|
| Baicalin | 80 | grams |
| Panax notoginseng | 150 | grams |
| EGCG | 150 | grams |
| Coriander extract | 70 | grams |
| Cardamom extract | 50 | grams |
| Chlorogenic acid | 70 | grams |

The resulting admixture is divided into 1,000 equal portions and each portion is placed into one of 1,000 appropriately-sized hard gelatine (or vegan) capsules.

### Example VIII

Capsules of EXAMPLE VII are administered in a dosing regimen of one capsule daily (e.g., with breakfast and dinner) to a subject at risk for infection by S. aureus. The treatment regimen is continued for 26 weeks.

### Example IX

The following ingredients are thoroughly admixed as close to uniform consistency as possible:

| | | |
|---|---|---|
| Rutin | 100 | grams |
| Hesperidin | 80 | grams |
| Sulforaphane | 2 | grams |
| Moringa leaf extract | 80 | grams |
| Rosmarinic acid | 6 | grams |
| Guava leaf extract | 20 | grams |
| Panax notoginseng | 150 | grams |

The resulting admixture is divided into 1,000 equal portions each placed into one of 1,000 appropriately-sized hard (or vegan) gelatin capsules.

### Example X

Capsules of EXAMPLE IX are administered in a dosing regimen of one capsule daily (e.g., with breakfast and dinner) to a subject at risk having viral infections. The treatment regimen is continued for 52 weeks. The described supplement supports the subject's health via, for example, a preventative well-care approach.

### Example XI

The following ingredients were thoroughly admixed to reduce heterogeneity:

| | | |
|---|---|---|
| Colostrum | 360 | g |
| Andrographis paniculata leaf extract | 100 | g |
| L-selenomethionine | 14 | g |
| Lactoferrin | 6.7 | g |
| Elderberry | 5 | g |
| Ground Clove powder | 33.30 | g |
| Rutin | 16.67 | g |
| Hesperidin | 20 | g |
| Scutellaria baicalensis root extract | 28.1 | g |
| Broccoli extract standardized | 3.3 | g |
| Moringa leaf extract powder | 26.67 | g |
| Rosemary powder extract | 22.22 | g |
| Guava leaf powder | 6.67 | g |
| Masgnesium stearate | 60 | g |

The resulting admixture is divided into 1,000 equal portions each placed into one of 1,000 appropriately-sized vegetarian capsules. For adult humans, the serving size of the capsules is three capsules.

### Example XII

Effect on Human Immune Response

INTRODUCTION: The immune system plays a critical role in defending the human body against infections. However, the human immune system ages over time and the aging process can be catalyzed by unhealthy decisions. Lack of exercise and poor diet can accelerate premature breakdown of the immune system—a process called “immuno-aging.” Weyh et al. “Physical Activity and Diet Shape the Immune System during Aging,” Nutrients 12, 622 (2020). The composition of EXAMPLE XI is an immune enhancing product that can support the human immune system through various mechanisms and help reduce the incidence of illness. The purpose of this study was to determine how the composition of EXAMPLE XI affects the immune system in human subjects.

METHODS: To test the ability of the composition of EXAMPLE XI to regulate the immune response, fifteen (15) individuals took the composition of EXAMPLE XI (three capsules by mouth daily) for three weeks. During the test period, participant saliva samples were collected at days 0, 7, and 21 in order to measure any change of various salivary biomarkers (e.g., immunoglobulins, interleukins, and TNF-α). Outliers were calculated and removed using an outlier calculator available at miniwebtool.com/outlier-calculator/.

RESULTS: The composition of EXAMPLE XI increases salivary SIgA and IL-10 levels. The data indicated that mucosal IgA (“SIgA”) showed an increasing trend throughout the three-week period, rising on average from less than about 200 micrograms/ml to about 225 micrograms/ml after three weeks. Interestingly, SIgA is a type of immunoglobulin A that acts as an antibody in the mucous membrane. It plays an important role in immune function not only in mucous, but also in tears, sweat, colostrum, and saliva. Low SIgA levels are usually related to reduced function of the immune system. Therefore, increased amounts of SIgA cause a stronger immune response.

In addition, higher levels of anti-inflammatory cytokine interleukin 10 (“IL-10”) in the saliva are observed, rising on average from about 0.65 picograms/ml to about 1.75 picograms/ml after three weeks. IL-10 plays an important role in balancing the inflammation status of the body to prevent acute inflammation and causing harm. Moreover, IL-10 also stimulates immune cells and antibody production. Mannino et al. “The paradoxical role of IL-10 in immunity and cancer.” Cancer Lett 367, 103-107 (2015). These results support the assertion that the composition of EXAMPLE XI can at least temporally increase the effectiveness of the immune response.

It was also determined that the composition of EXAMPLE XI did not affect the levels of other pro-inflammatory cytokines in the subjects tested. Specifically, it was investigated whether the composition of EXAMPLE XI globally increases salivary cytokines levels. The resulting data showed that the composition of EXAMPLE XI does not upregulate all cytokines levels. Instead, levels of some pro-inflammatory cytokines like tumor necrosis factor alpha (TNF-α) and interleukin 8 (IL-8) remain unchanged (data not shown), suggesting that the composition of EXAMPLE XI regulates a specific pool of cytokines that can promote immune health.

CONCLUSION: Supplementation with the herein described compositions increase salivary SIgA and IL-10 levels in human subjects, suggesting that the described compositions have the ability to promote function of the human immune system by regulating the expression levels of different cytokines. The increase of cytokines is usually observed during infections, however, the described composition slightly boosts SIgA and IL-10 levels even without bacterial or viral infections, suggesting it plays a role in preparing the immune system to fight against such infections. Furthermore, this modulation is only applied to specific cytokines, since the data showed that the composition did not alter levels of different pro-inflammatory cytokines. Combining the in vitro and in vivo observations, these results demonstrate the ability of the described composition to support better immunity in humans by modulating the immune system.

### Example XIII

Evaluation of the Composition of EXAMPLE XI for Anti-Quorum Sensing and Anti-Biofilm Activity

BACKGROUND: The human body is a living ecosystem; for every human cell in the body there are 10 bacterial cells that perform different functions. Both “good” and “bad” bacteria exist inside the human gut and are constantly battling for surface area. A large subset of pathogenic bacteria can compromise overall health and, specifically, immune health in the body. The ability of bad bacteria of different species to “talk” to each other, increase in growth, become virulent, and cause infectious disease is due to a process called “quorum sensing”. This study assessed the ability of a model product, the composition of EXAMPLE XI, to inhibit quorum sensing in C. violaceum. C. violaceum is a good model system for studying quorum sensing because the small molecule this bacterium employs to “talk” with other bacteria is violet in color and can easily be detected.

SUMMARY OF METHODS: (+)-Catechin was used as a positive control in the halo and violaceum assays. Varying concentrations of the composition of EXAMPLE XI or catechin mixed with C. violaceum were added to an agar plate and the generated halo was analyzed both by visual inspection and measurement. Method for Violaceum assays included adding either the composition of EXAMPLE XI or catechin to a solution of C. violaceum (strain CV026). After workup, the solutions were transferred to a 96 well plate and optical density was recorded at 590 nm. Optical density values were normalized to unextracted samples as a control.

SUMMARY OF RESULTS: Halo assays found that the composition of EXAMPLE XI inhibited C. violaceum growth more effectively than the positive control, and did so via a quorum sensing inhibition mode of action rather than through a traditional antibiotic action as Catechin does. The violaceum assay confirmed that the described model composition inhibited quorum sensing by over 90% at a concentration of 5 mg/ml.

SUMMARY OF CONCLUSION: These results demonstrate the ability of the model composition to inhibit bacterial growth by inhibiting quorum sensing.

GOAL: Evaluate the described compositions for anti-quorum sensing and anti-biofilm activities. Towards this end, the model composition (the composition of Example XI) was examined for its ability to generate a Halo on Chromobacterium violaceum (CV026) plates; affect violaceum (C. violaceum) and pyocyanin (P. aeruginosa) production and possess antibiofilm activity (P. aeruginosa and S. aureus).

Materials and Methods

Test compounds: The composition of EXAMPLE XI was used as capsules and in powder form. Catechin (+)—was purchased from Sigma-Aldrich (catalog number C1251).

Stock solutions: The composition of EXAMPLE XI powder and (+)—Catechin were dissolved/suspended in DMSO or ethanol, to a concentration of 100 mg/mL. Further working stocks were made by preparing dilutions with DMSO or ethanol.

Bacteria: Pseudomonas aeruginosa and Staphylococcus aureus were obtained from the American Type Culture Collection, catalog numbers 10145 and 12600, respectively. Chromobacterium violaceum strain CV026 was obtained from Dr. Robert McLean in the Department of Biology at Texas State University, San Marcos, Tex., US.

Halo Assay: Halo assays were performed based on the procedure of Chong, et al., Journal of Natural Products 2011, 74, pp. 2261-2264. Briefly, a fresh overnight culture of C. violaceum (CV026) was prepared and adjusted to an optical density at 600 nm (OD600) of 0.8 in Tryptic Soy broth (“TSB”). Top agar consisting of TSB and 0.8% agar was melted and equilibrated to 50 degrees centigrade. To 5 mL of top agar, N-3-oxohexanoyl-homoserine lactone (C6-HSL) was added to a final concentration of 0.25 mg/mL and 200 microliters of the diluted CV026 (OD600=0.8) was added. The mixture was vortexed and poured onto a 100 mm petri plate containing TSB/1% agar. The plate was swirled to produce an even distribution of the top agar. The plate was equilibrated to room temperature. Subsequently, ten microliters of the model composition or Catechin in DMSO was applied to the surface of the plate at several different concentrations. Ten microliters of DMSO was also applied as a control. The plate was incubated for 24 hours at 30 degrees C.

Violaceum Assay: The violaceum assay was adapted from the method of Blosser, et al., Journal of Microbiological Methods 2000, 40, pp. 47-55. The model composition and Catechin were suspended/dissolved in ethanol. Aliquots of each were dispensed in duplicate into 25×75 mm snap-cap culture tubes to produce final concentration in 2 mL, of 5, 1.25, 0.5 and 0.25 mg/mL. The tubes were placed in a rotary evaporator until dry. A fresh overnight culture of C. violaceum (CV026) was prepared and adjusted to an optical density at 600 nm (OD600) of 0.1 in TSB. C6-HSL was added to a final concentration of 0.5 ug/mL. Two mL of the dilute CV026 was added to each of the culture tubes and the tubes were shaken overnight at 30 degrees C. The OD600 of each culture was measured to be used to normalize violaceum levels to total biomass. Two hundred microliters of each culture was combined with 200 microliters of 10% sodium dodecyl sulfate in a 1.5 mL microfuge tube. The mixtures were vortexed and incubated at room temperature for 10 minutes to lyse the bacteria. Six hundred microliters of water-saturated butanol was added and the tubes were vortexed for 30 seconds. The tubes were centrifuge at 5,000 g for 5 minutes. Two hundred microliters of the butanol phase was transferred to individual wells of a 96-well flat-bottom polystyrene plate. The optical density was measured at 590 nm. The OD590 values were normalized to the biomass measurements determined for the unextracted samples.

Pyocyanin Assay: The violaceum assay was adapted from the method of Tan, et al., Sensors 2012, 12, pp. 4339-4351. The assay was performed using suspensions/solutions of the model composition and catechin in DMSO and in ethanol. 5, 10, 25 and 100 mg/mL suspensions/solutions of the model composition and Catechin in DMSO and ethanol were prepared. 200 microliters of each solution was added to duplicate 15 mL conical tubes. Also 200 microliters of DMSO or ethanol were added to duplicate tubes for the vehicle controls. For the tubes in which the test materials were in ethanol, they were dried using a rotary evaporator. A fresh overnight culture of Pseudomonas aeruginosa (ATCC 10145) was prepared and adjusted to an OD600 of 0.2 in Nutrient broth. 4 mL of the culture was added to all of the tubes receiving the test compounds in ethanol, and 3.8 mL of the culture to those tubes receiving the test substance in DMSO. The samples were incubated overnight, with shaking at 37 degrees C. After the overnight incubation, four milliliters of chloroform were added to all tubes, and the tubes were vortexed for 20 seconds four times, and centrifuged at 5,000 g for 5 minutes. The aqueous phase was removed. To the chloroform phase, 1 mL of 0.2 N HCl was added and the tubes were vortexed several times. Two hundred microliters of the aqueous phase was pipetted into individual wells of a flat-bottom, 96-well polystyrene plate. The amount of pyocyanin in each well was determined by measuring the optical density at 520 nm.

Biofilm assay: The biofilm assay was based on the method of Chong, et al. (2011). The model composition and Catechin were prepared at 100 mg/mL in ethanol. Appropriate volumes of each were added to eight replicate wells of a v-well 96-well plate to result in 0.25, 0.5, 1.25 and 5 mg/mL final concentrations for a 200 microliter final volume. The plate was air dried until all of the ethanol has evaporated. From fresh overnight cultures of P. aeruginosa (ATCC 10145) and S. aureus (ATCC 12600), solutions at OD600 of 0.1 were prepared in Nutrient broth/0.5% glucose. Two hundred microliters of the cultures were added to all wells and mixed by pipetting. The plates were covered with breathable films and incubated overnight without shaking at 37 degrees C. The cultures were removed, and the plates rinsed several times with water. Two hundred microliters of 0.1% crystal violet was added to each well and incubated at room temperature for 30 minutes. The crystal violet was removed, and the plates rinsed four times with water. The plates were air dried for 3 hours. The crystal violet was solubilized with 200 microliters of 95% ethanol. One hundred microliters of the solubilized crystal violet was transferred to a flat-bottom polystyrene plate and the optical density measured at 590 nm.

Results and Discussion:

HALO ASSAY: The Halo assay is often used to evaluate anti-quorum sensing activity. In Gram-negative bacteria quorum-sensing is mediated by soluble signaling molecules, usually N-acylhomoserine lactones (“AHLs”). Chromobacterium violaceum is a useful model system for studying quorum sensing since production of the violet pigment, violaceum is induced in response to N-3-oxohexanoyl-homoserine lactone (C6-HSL). CV026, a mutant strain of C. violaceum has been isolated for use as a biosensor for quorum sensing activity (McClean et al., Microbiology 1997, 143, 3703-3711). CV026 does not produce ALHs, consequently, it requires exogenous administration of AHLs to induce a quorum sensing response. The composition of EXAMPLE XI was evaluated for anti-quorum sensing activity in a Halo assay using the biosensing bacterial strain C. violaceum (CV026). As a positive control, Catechin was also evaluated.

The resulting halos produced by the composition of EXAMPLE XI were of comparable or greater size than those of equivalent amounts of the positive control, Catechin. For the highest concentrations of Catechin, the halos exhibit a transparency that suggests that the clearing is not due simply to the inhibition of violaceum production, but due to an antibiotic effect inhibiting growth of the bacteria. In contrast, the model composition inhibited violaceum production, but did not significantly inhibit bacterial growth.

VIOLACEUM ASSAY: The Violaceum assay was performed to further corroborate the results obtained in the Halo assay and to provide a quantitative measure of activity. This assay is based on the same principles as for the Halo assay, but since the assay is performed in solution, violaceum production can be more readily quantified. The Violaceum assay was initially performed as described by Tan, et al. (2012). For this, C. violaceum (CV026) was dispensed into individual wells of a 96-well plate. Test substances were added at varying concentrations and the plate was incubated overnight at 30 degrees C. Subsequently, the plated was placed at 60 degrees C. until dry. Finally, DMSO was added to solubilize the dried material and the optical density was measured at 590 nm.

It was clear from the results that the approach had two problems. First, the vehicle (DMSO) alone inhibited violaceum production. As a result, it would not be possible to use DMSO as the solvent for preparing the model composition XI and Catechin test substances. Second, both the model composition and Catechin impart coloration to the test cultures and can add to the measured optical density at 590 nm. The absence of an effect of DMSO on the Halo assay may be due to the DMSO diffusing into the agar and becoming too dilute to inhibit violaceum production in the plate-based assay.

To deal with the fact that the colored samples interfere with the optical density measurements, an alternative Violaceum assay was performed based on that reported by Blosser, et al. (2000). The important difference is that the optical density of the assay solutions is not measured directly. Instead, the assay solutions are extracted with butanol and the optical density of the extracts is measured. In this way, the violaceum is extracted from the assay samples and colored components of the test samples are not. Furthermore, the test samples were suspended/dissolved in ethanol instead of DMSO.

Based on the modified Violaceum assay, both the model composition and Catechin inhibit violaceum production at the highest concentration tested for each test substance. Violaceum production was inhibited by over 90% for the model composition and over 85% for Catechin. At 1.25 and 0.5 mg/mL, the model composition appears to be inhibiting violaceum production by close to 20%, whereas violaceum production is at untreated levels by 0.25 mg/mL. Inhibition of violaceum production by Catechin at the three lowest concentrations tested ranges from 10-25%.

PYOCYANIN ASSAY: Pyocyanin is a toxin produced by Pseudomonas aeruginosa for the purpose of killing competing microbes. Pyocyanin is also toxic to mammalian cells and contributes to the pathogenicity in, e.g., cystic fibrosis patients with P. aeruginosa pulmonary infections. Pyocyanin is a blue in color allowing P. aeruginosa strains producing it to be used as biosensors in assays aimed at evaluating a substances ability to defeat one of its defenses. The model composition was evaluated for its ability to inhibit pyocyanin production. For this, cultures of P. aeruginosa were incubated overnight with varying concentrations of the model composition and Catechin. Test substances are generally dissolved/suspended in DMSO because it is such a universal solvent. Because of the difficulty encountered using DMSO for the violaceum assay, test materials prepared in DMSO and ethanol were examined.

The composition of EXAMPLE XI had no activity inhibiting pyocyanin production by P. aeruginosa. In fact, it slightly enhances production at the highest concentrations tested, with production returning to untreated levels at the lower concentrations. Similarly, Catechin slightly enhances production of pyocyanin. This is in contrast to published reports by Chong, et al. (2011). The choice of solvent does not alter the results for the model composition.

BIOFILM ASSAY: The composition of EXAMPLE XI was evaluated for anti-biofilm activity against Pseudomonas aeruginosa and Staphylococcus aureus. For this, cultures of P. aeruginosa and S. aureus were dispensed into microtiter plates. The composition of EXAMPLE XI and Catechin were added at varying concentrations.

The plates were incubated statically overnight. The cultures were removed, the plate rinsed several times and then stained with crystal violet. After rinsing, and drying the plate, the remaining crystal violet was solubilized with 95% ethanol. The crystal violet was quantified by measuring the optical density at 590 nm.

The composition of EXAMPLE XI has no anti-biofilm activity against P. aeruginosa or S. aureus. The activity is similar for Catechin.

CONCLUSION: This EXAMPLE shows that the described compositions are able to inhibit bacterial growth of C. violaceum as seen in halo assay experiments by the mode of action of inhibiting quorum sensing.

### REFERENCES

### The Contents of Each of which are Incorporated Herein by this Reference

Al-Haidari et al., “Anti-Quorum Sensing Activity of Some Medicinal Plants,” Afr. Tradit. Complement Ahern. Med. (2016) 13(5):67-71. doi:10.21010/ajtcam.v13i5.10.
Blosser, et al., Journal of Microbiological Methods 2000, 40, pp. 47-55.
Chang et al., “Non-antibiotic quorum sensing inhibitors acting against N-acyl homoserine lactone synthase as druggable target,” Nature Scientific Reports 4:7245 (2014); DOI: 10.1038/srep07245.
Chen et al., “1H NMR-Based Global Metabolic Studies of Pseudomonas aeruginosa upon Exposure of the Quorum Sensing Inhibitor Resveratrol,” J. Proteome Res. 2017, 16, 824-830. DOI: 10.1021/acs.jproteome.6b00800.
Chong et al., “Malabaricone C from Myristica cinnamomea Exhibits Anti-Quorum Sensing Activity,” J. Nat. Prod. 2011, 74, 2261-2264. dx. doi. org/10.1021/np100872k.
Choo et al., “Inhibition of bacterial quorum sensing by vanilla extract,” Letters in Applied Microbiology 42: 637-641 (2006).
Dolgin, Elie “The secret social lives of viruses,” Nature 2019, 570 (7761): 290-292. doi:10.1038/d41586-019-01880-6.
Ghosh et al., “Guava Leaf Extract Inhibits Quorum-Sensing and Chromobacterium violaceum Induced Lysis of Human Hepatoma Cells: Whole Transcriptome Analysis Reveals Differential Gene Expression,” PLOS ONE 9(9):1-12 e107703 (2014).
Haripriyan et al., “Clove Bud Oil Modulates Pathogenicity Phenotypes of the Opportunistic Human Pathogen Pseudomonas aeruginosa,” Nature Scientific Reports 8:3437 (2018); DOI: 10.1038/s41598-018-19771-7.
Jakobsen et al., “Ajoene, a Sulfur-Rich Molecule from Garlic, Inhibits Genes Controlled by Quorum Sensing,” Antimicrobial Agents and Chemotherapy 56(5):2314-2325 (2012).
Jakobsen et al., “Food as a Source for Quorum Sensing Inhibitors: Iberin from Horseradish Revealed as a Quorum Sensing Inhibitor of Pseudomonas aeruginosa,” Applied and Environmental Microbiology 78(7):2410-2421 (2012).
Koh et al., “Plant-Derived Natural Products as Sources of Anti-Quorum Sensing Compounds,” Sensors 2013, 13, 6217-6228; doi:10.3390/s130506217.
Lou et al., “The effect of burdock leaf fraction on adhesion, biofilm formation, quorum sensing and virulence factors of Pseudomonas aeruginosa,” Journal of Applied Microbiology 122, 615-624 (2016).
Mannino et al. “The paradoxical role of IL-10 in immunity and cancer.” Cancer Lett 367, 103-107 (2015).
Muhs et al., “Virulence Inhibitors from Brazilian Peppertree Block Quorum Sensing and Abate Dermonecrosis in Skin Infection Models,” Nature Scientific Reports 7:42275 (2017); DOI: 10.1038/srep42275.
Ta et al., “Mini Review of Phytochemicals and Plant Taxa with Activity as Microbial Biofilm and Quorum Sensing Inhibitors,” Molecules 2016, 21, 29; doi:10.3390/molecules21010029.
Tan, et al., Sensors 2012, 12, pp. 4339-4351.
Tan et al., “Comparative Systems Biology Analysis To Study the Mode of Action of the Isothiocyanate Compound Iberin on Pseudomonas aeruginosa,” Antimicrobial Agents and Chemotherapy 58(11): 6648-6659 (November 2014).
Vandeputte et al., “The flavanone naringenin reduces the production of quorum sensing-controlled virulence factors in Pseudomonas aeruginosa PAO1,” Microbiology (2011), 157, 2120-2132.
Vasavi et al., “Anti-quorum sensing activity of Psidium guajava L. flavonoids against Chromobacterium violaceum and Pseudomonas aeruginosa PAO1,” Microbiol. Immunol. 2014; 58: 286-293; doi: 10.1111/1348-0421.12150.
Verma et al., “Quorum sensing in the squid-Vibrio symbiosis,” Int. J. Mol. Sci. 14(8):16386-401 (2013); doi: 10.3390/ijms140816386.
Weyh et al. “Physical Activity and Diet Shape the Immune System during Aging,” Nutrients 12, 622 (2020).
Wu et al., “Natural Products That Target Virulence Factors in Antibiotic-Resistant Staphylococcus aureus,” J Agric. Food Chem. 2019, 67, 13195-13211. DOI: 10.1021/acs.jafc.9b05595.
Yin et al., “Tea polyphenols as an antivirulence compound Disrupt Quorum-Sensing Regulated Pathogenicity of Pseudomonas aeruginosa,” Nature Scientific Reports 5:16158 (2015); DOI: 10.1038/srep16158.

What is claimed is:

1. A dosage form for administration to a subject, the dosage form comprising at least six ingredients selected from the group consisting of clove powder, rutin, hesperidin, baicalin, sulforaphane, moringa leaf extract, rosmarinic acid, guava leaf extract and/or powder, Panax notoginseng, EGCG, propolis, coriander extract, cardamom extract, cinnamaldehyde, chlorogenic acid, caper fruit, vescalagin, nutmeg extract, and naringenin.

2. The dosage form of claim 1, wherein the dosage form contains amounts of the selected at least six ingredients to inhibit quorum sensing by bacteria, fungi, or viruses as may be determined by a halo assay test.

3. The dosage form of claim 2, wherein the selected at least six ingredients, when present, are present in the following amounts:
from about 25 to about 200 milligrams of clove powder,
from about 30 to about 100 milligrams of rutin,
from about 40 to about 80 milligrams of hesperidin,
from about 60 to about 100 milligrams of baicalin,
from about 0.08 to about 2 milligrams of sulforaphane,
from about 50 to about 120 milligrams of moringa leaf extract,
from about 4 to about 8 milligrams of rosmarinic acid,
from about 10 to about 30 milligrams of guava leaf extract and/or powder,
from about 75 to about 200 milligrams of Panax notoginseng,
from about 75 to about 160 milligrams of EGCG,
from about 20 to about 40 milligrams propolis,
from about 50 to about 90 milligrams of coriander extract,
from about 30 to about 60 milligrams of cardamom extract,
from about 2 to about 5 milligrams of cinnamaldehyde,
from about 50 to about 90 milligrams of chlorogenic acid,
from about 20 to about 40 milligrams of caper fruit,
from about 3 to about 8 milligrams of vescalagin,
from about 10 to about 150 milligrams of nutmeg extract, and
from about 10 to about 30 milligrams of naringenin.

4. The dosage form of claim 1, wherein the at least six ingredients are selected from the group consisting of rutin, hesperidin, baicalin, sulforaphane, rosmarinic acid, cinnamaldehyde, chlorogenic acid, and naringenin.

5. The dosage form of claim 1, wherein the dosage form is selected from the group consisting of a softgel, capsule, tablet, gel, powder, gummy, liquid, effervescent, bar, topical patch, serum, stick pack, ready to drink composition, lotion, and cream.

6. The dosage form of claim 1, wherein the dosage form further comprises other nutraceutical raw ingredients known to support health.

7. The dosage form of claim 6, wherein the other nutraceutical raw ingredients include colostrum, arabinogalactan, lactoferrin, zinc, and/or elderberry.

8. A method of inhibiting quorum sensing by at least one infectious agent in a subject, the method comprising:
administering the dosage form of claim 2 to the subject in an amount effective to inhibit quorum sensing by the at least one infectious agent in the subject.

9. A method of inhibiting quorum sensing by an infectious agent in a subject, the method comprising:
administering a combination of at least six selected ingredients to the subject, wherein the at least six ingredients are selected from the group consisting of clove powder, rutin, hesperidin, baicalin, sulforaphane, moringa leaf extract, rosmarinic acid, guava leaf extract and/or powder, Panax notoginseng, EGCG, propolis, coriander extract, cardamom extract, cinnamaldehyde, chlorogenic acid, caper fruit, vescalagin, nutmeg extract, and Naringenin.

10. The method according to claim 9, wherein the subject ingests the selected at least six ingredients, when selected, in the following amounts on a daily basis:
from about 25 to about 200 milligrams of clove powder,
from about 30 to about 100 milligrams of rutin,
from about 40 to about 80 milligrams of hesperidin,
from about 60 to about 100 milligrams of baicalin,
from about 0.08 to about 2 milligrams of sulforaphane,
from about 50 to about 120 milligrams of moringa leaf extract,
from about 4 to about 8 milligrams of rosmarinic acid,
from about 10 to about 30 milligrams of guava leaf extract and/or powder,
from about 75 to about 200 milligrams of Panax notoginseng,
from about 75 to about 160 milligrams of EGCG,
from about 20 to about 40 milligrams propolis,
from about 50 to about 90 milligrams of coriander extract,
from about 30 to about 60 milligrams of cardamom extract,
from about 2 to about 5 milligrams of cinnamaldehyde,
from about 50 to about 90 milligrams of chlorogenic acid,
from about 20 to about 40 milligrams of caper fruit,
from about 3 to about 8 milligrams of vescalagin,
from about 10 to about 150 milligrams of nutmeg extract, and
from about 10 to about 30 milligrams of naringenin.

11. The method according to claim 10, for use in inhibiting quorum sensing by an infectious agent selected from the group consisting of bacteria, viruses, fungi, protozoa, and helminths.

12. The method according to claim 11, wherein the infectious agent is a bacterium selected from the group consisting of Pseudomonas aeruginosa, Escherichia coli, Chromobacterium violaceum, Pseudomonas fluorescens, Staphylococcus aureus, Helicobacter pylori, Candida albicans, Streptococcus mutans, Bacillus cereus, Proteus mirabilis, Serratia marcescens, Acinetobacter, Burkholderia, and any combination thereof.

13. A method of making the dosage form of claim 1, the method comprising admixing the selected ingredients and associating them together into or with the dosage form.

14. A composition of ingredients including plant secondary metabolites having the ability to inhibit quorum sensing in both bacteria and viruses responsible for causing disease in humans.

15. A method of promoting health of a subject's immune system, the method comprising:
administering the dosage form of claim 1 to the subject in an amount effective to increase salivary mucosal IgA (“SIgA”) and IL-10 levels in the subject.

16. The method according to claim 15, wherein the subject ingests the selected at least six ingredients, when selected, in the following amounts on a daily basis:
from about 25 to about 200 milligrams of clove powder,
from about 30 to about 100 milligrams of rutin,
from about 40 to about 80 milligrams of hesperidin,
from about 60 to about 100 milligrams of baicalin,
from about 0.08 to about 2 milligrams of sulforaphane,
from about 50 to about 120 milligrams of moringa leaf extract,
from about 4 to about 8 milligrams of rosmarinic acid,
from about 10 to about 30 milligrams of guava leaf extract and/or powder,
from about 75 to about 200 milligrams of Panax notoginseng,
from about 75 to about 160 milligrams of EGCG,
from about 20 to about 40 milligrams propolis,
from about 50 to about 90 milligrams of coriander extract,
from about 30 to about 60 milligrams of cardamom extract,
from about 2 to about 5 milligrams of cinnamaldehyde,
from about 50 to about 90 milligrams of chlorogenic acid,
from about 20 to about 40 milligrams of caper fruit,
from about 3 to about 8 milligrams of vescalagin,
from about 10 to about 150 milligrams of nutmeg extract, and
from about 10 to about 30 milligrams of naringenin.

17. A method of promoting health of a subject's immune system, the method comprising:
administering a combination of at least six selected ingredients to the subject, wherein the at least six selected ingredients are selected from the group consisting of clove powder, rutin, hesperidin, baicalin, sulforaphane, moringa leaf extract, rosmarinic acid, guava leaf extract and/or powder, Panax notoginseng, EGCG, propolis, coriander extract, cardamom extract, cinnamaldehyde, chlorogenic acid, caper fruit, vescalagin, nutmeg extract, and Naringenin.

18. The method according to claim 17, wherein the subject ingests the selected at least six ingredients, when selected, in the following amounts on a daily basis:
from about 25 to about 200 milligrams of clove powder,
from about 30 to about 100 milligrams of rutin,
from about 40 to about 80 milligrams of hesperidin,
from about 60 to about 100 milligrams of baicalin,
from about 0.08 to about 2 milligrams of sulforaphane,
from about 50 to about 120 milligrams of moringa leaf extract,
from about 4 to about 8 milligrams of rosmarinic acid,
from about 10 to about 30 milligrams of guava leaf extract and/or powder,
from about 75 to about 200 milligrams of Panax notoginseng,
from about 75 to about 160 milligrams of EGCG,
from about 20 to about 40 milligrams propolis,
from about 50 to about 90 milligrams of coriander extract,
from about 30 to about 60 milligrams of cardamom extract,
from about 2 to about 5 milligrams of cinnamaldehyde,
from about 50 to about 90 milligrams of chlorogenic acid,
from about 20 to about 40 milligrams of caper fruit,
from about 3 to about 8 milligrams of vescalagin,
from about 10 to about 150 milligrams of nutmeg extract, and
from about 10 to about 30 milligrams of naringenin.
